(19) United States
(12) Patent Application Publication
(10) Pub. No.: US 2025/0381075 A1
(43) Pub. Date: Dec. 18, 2025

(54) INCONTINENCE DETECTION

(71) Applicant: Hill-Rom Services, Inc., Batesville, IN (US)

(72) Inventors: Heinz-Hermann Dalbert, Charleston, SC (US); Nicholas Mann, Cincinnati, OH (US); Todd O'Neal, Fairfield, OH (US)

(21) Appl. No.: 19/240,214

(22) Filed: Jun. 17, 2025

Related U.S. Application Data
(60) Provisional application No. 63/661,352, filed on Jun. 18, 2024.

(51) Int. Cl. A61F 13/42

(52) U.S. Cl. CPC A61F 13/42; A61F 2013/424

(57) ABSTRACT

A mattress includes one or more internal structures, an outer cover surrounding the one or more internal structures, and a sensor fixed between the one or more internal structures and the outer cover such that the sensor is integrated with the mattress. The sensor includes a substrate and one or more electrically conductive traces arranged on the substrate. The one or more electrically conductive traces generate an electrical field that is interrupted by a presence of a fluid. The sensor further includes an electrical connector for connecting the one or more electrically conductive traces to a controller housed on a frame that is configured to support the mattress. An interruption of the electrical field causes the controller to issue an alert when the presence of the fluid outside of the mattress exceeds a threshold.

### CROSS-REFERENCE TO RELATED APPLICATION

This application claims the benefit of and priority to U.S. Provisional Patent Application No. 63/661,352, filed June 18, 2024, the entire disclosure of which is incorporated by reference herein in its entirety.

### BACKGROUND

Urinary incontinence, also known as involuntary urination, is any uncontrolled leakage of urine. It is a common and distressing problem, which may have a large impact on quality of life. Incontinence is typically exhibited by bedridden patients, geriatric patients, and pediatric patients who experience bed wetting. Urinary incontinence can also result from an underlying medical condition or operation such as pelvic surgery, pregnancy, childbirth, and menopause. Incontinence is an example of a stigmatized medical condition in which patients may be too embarrassed to seek medical help, and attempt to self-manage the symptom.

The stigmatization associated with incontinence can create barriers to successful management of incontinence which can lead to incontinence-associated dermatitis (IAD). Incontinent patients are also more likely to develop pressure injuries than continent patients because skin can be compromised in as little as 15 minutes after exposure to moisture. Incontinence can also negatively impact a patient's mental health and quality of life. Improving the patient experience and reducing skin breakdown and pressure injuries can have measurable impacts on patient satisfaction, clinical outcomes, and hospital economics.

### SUMMARY

In general terms, the present disclosure relates to incontinence detection. In one configuration, a sensor for detecting incontinence is integrated with a mattress. Various aspects are described in this disclosure, which include, but are not limited to, the following aspects.

One aspect relates to a mattress comprising: one or more internal structures; an outer cover surrounding the one or more internal structures; and a sensor fixed between the one or more internal structures and the outer cover such that the sensor is integrated with the mattress, the sensor including: a substrate; one or more electrically conductive traces arranged on the substrate, the one or more electrically conductive traces configured to generate an electrical field that is interrupted by a presence of a fluid; and an electrical connector for connecting the one or more electrically conductive traces to a controller housed on a frame that is configured to support the mattress, wherein an interruption of the electrical field causes the controller to issue an alert when the presence of the fluid outside of the mattress exceeds a threshold.

Another aspect relates to a method of detecting incontinence, the method comprising: generating an electrical field using a sensor integrated with a mattress; detecting an interruption of the electrical field; determining whether the interruption of the electrical field exceeds a threshold such that a fluid outside of the mattress exceeds a predetermined amount; and issuing an alert when the interruption of the electrical field exceeds the threshold.

Another aspect relates to a sensor for detecting incontinence, the sensor comprising: a substrate configured to be fixed between one or more internal structures of a mattress and an outer cover of the mattress for integration with the mattress; one or more electrically conductive traces applied on the substrate, the one or more electrically conductive traces configured to generate an electrical field that is interrupted by a presence of fluid outside of the mattress; and an electrical connector configured to connect the one or more electrically conductive traces to a controller housed on a frame supporting the mattress, wherein an interruption of the electrical field causes the controller to issue an alert indicating the presence of the fluid outside of the mattress exceeds a threshold volume.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for detecting incontinence of a patient resting on a patient support apparatus inside a patient environment.

FIG. 2 is an isometric view of a mattress of the patient support apparatus of FIG. 1 with a cutaway portion exposing an interior of the mattress.

FIG. 3 illustrates an example of a sensor integrated inside the mattress of FIG. 2.

FIG. 4 illustrates another example of the sensor that can be integrated inside the mattress of FIG. 2.

FIG. 5 illustrates another example of the sensor that can be integrated inside the mattress of FIG. 2.

FIG. 6 illustrates another example of the sensor that can be integrated inside the mattress of FIG. 2.

FIG. 7 schematically illustrates an example of a method of detecting incontinence that can be performed by a controller of the patient support apparatus of FIG. 1.

FIG. 8 schematically illustrates an incontinence page that can be generated based on the data collected by the controller of the patient support apparatus of FIG. 1.

FIG. 9 schematically illustrates an example of the controller that can be housed on a frame of the patient support apparatus of FIG. 1.

FIG. 10 is an exploded isometric view of an example of a microclimate management assembly of the mattress of FIG. 2 that includes an example implementation of the sensor of FIG. 3 or 4 for detecting incontinence.

### DETAILED DESCRIPTION

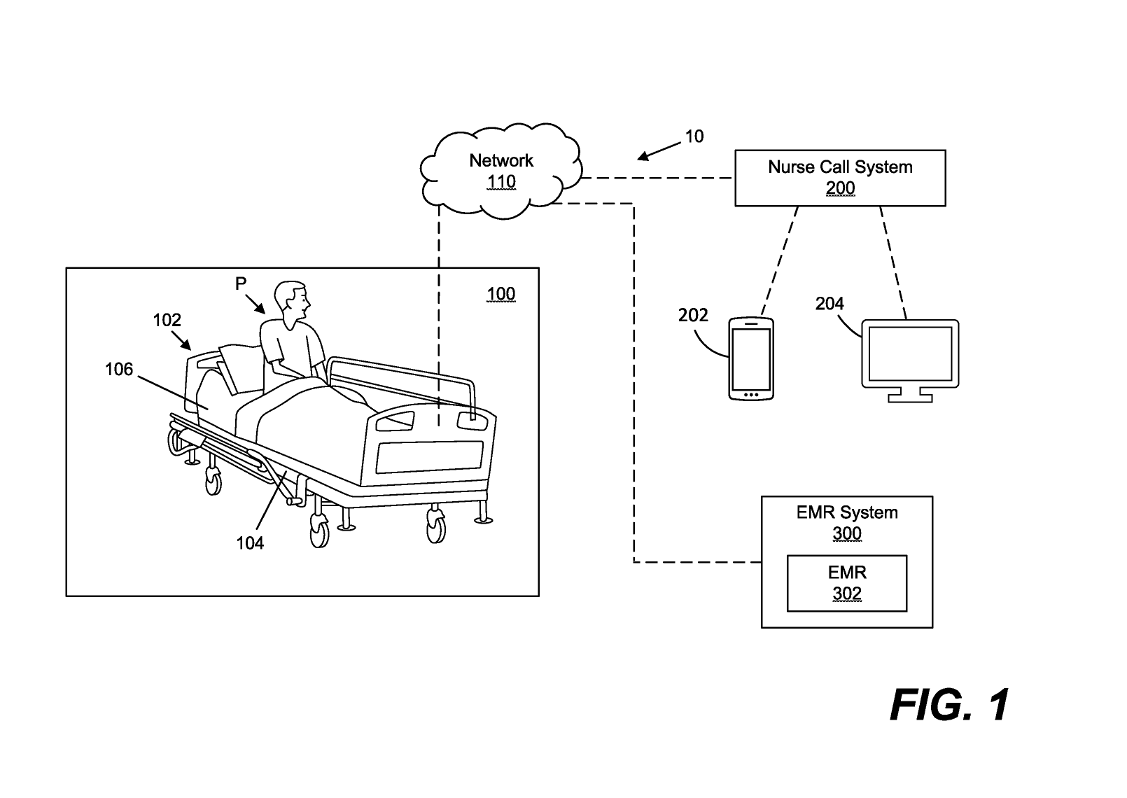

FIG. 1 illustrates an example of a system 10 for detecting incontinence of a patient P resting on a patient support apparatus 102 inside a patient environment 100. As will be described in more detail further below, the system 10 aids in early incontinence detection which can lead to better skin outcomes for the patient P. For example, once incontinence is detected, an alert is generated for a caregiver to assist the patient P such as by assisting the patient P to a bathroom, changing the patient P's clothing, changing linens and sheets on the patient support apparatus 102, and other actions to mitigate the patient P's exposure to moisture.

The patient environment 100 is an area within a healthcare facility such as a patient room in a hospital. Examples of the patient support apparatus 102 can include a hospital bed, or similar type of apparatus on which the patient P rests while admitted in the healthcare facility.

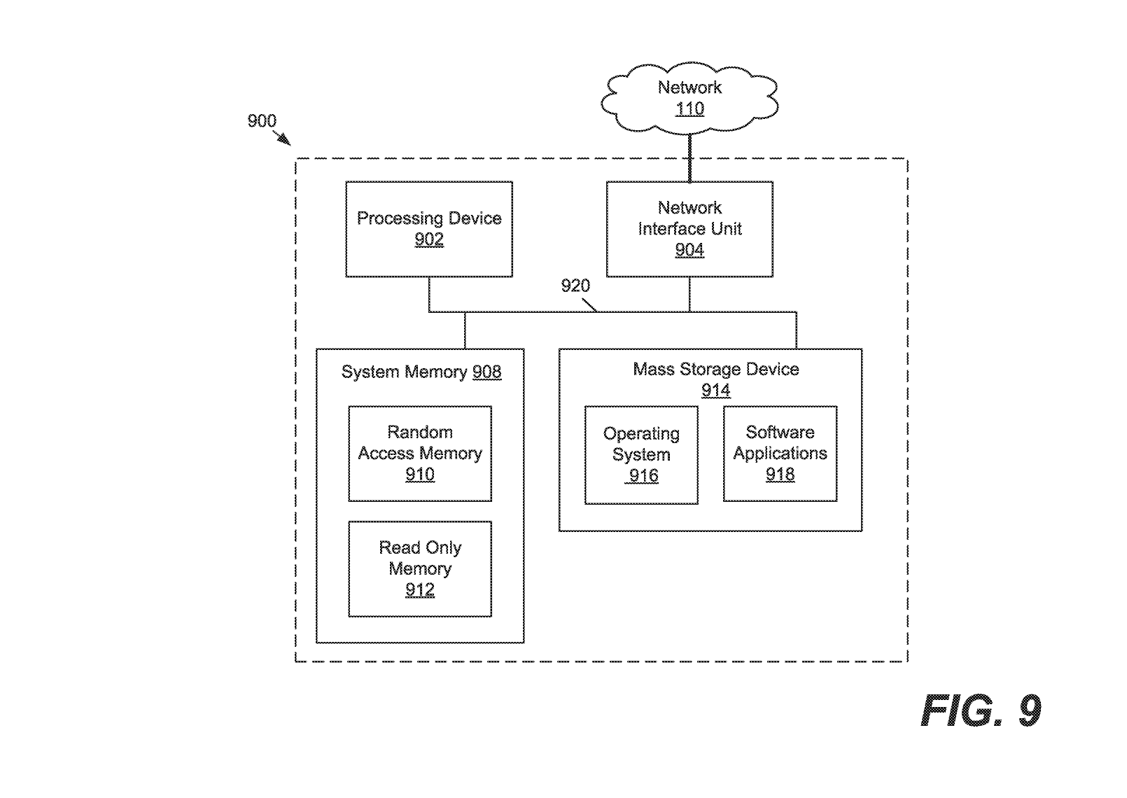

As shown in FIG. 1, the patient support apparatus 102 includes a mattress 106 that is supported on a frame 104. The frame 104 can be used to adjust a height of the mattress 106 relative to the ground, and can be articulated to adjust an angle or inclination of an upper portion of the mattress 106 relative to a lower portion of the mattress. The frame 104 can include one or more motors for adjusting the height and angle of the mattress 106, and can further include a controller 900 (see FIG. 9) for controlling the motors as well as for receiving physiological parameter data and alerts from one or more sensors of the patient support apparatus 102.

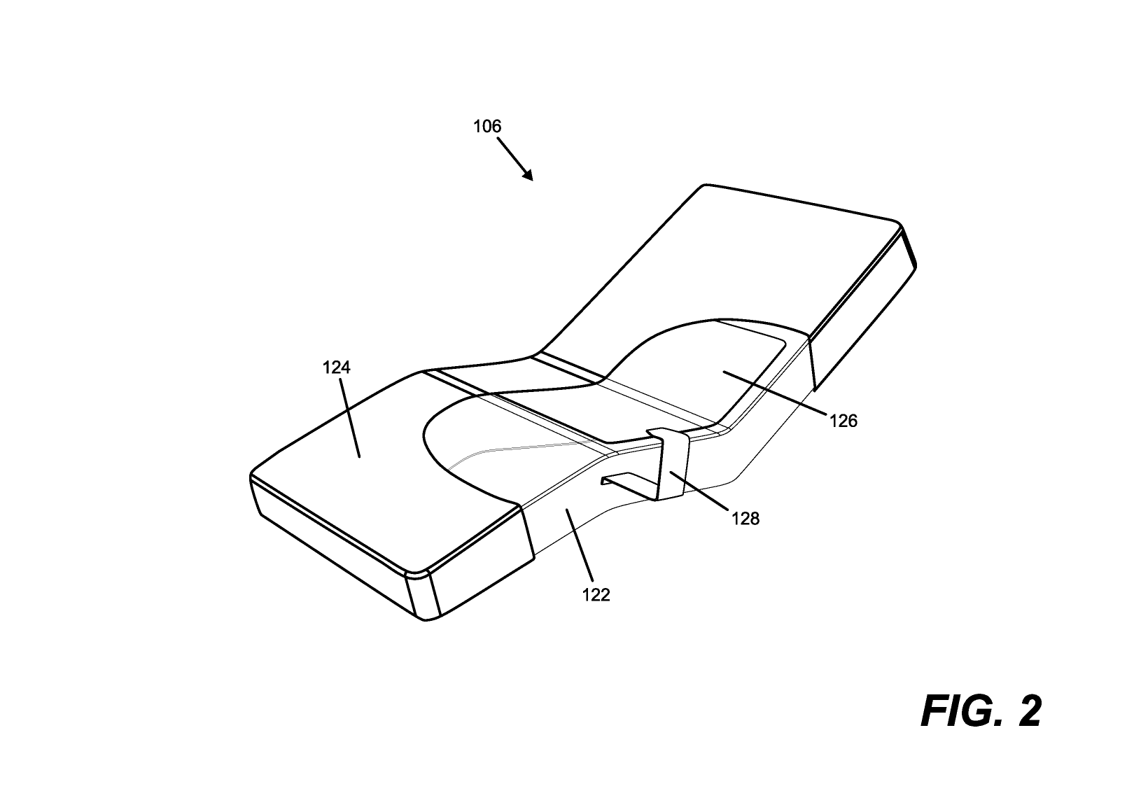

The patient support apparatus 102 further includes a sensor 126 (see FIG. 2) that detects a presence of fluid or moisture without contacting the fluid or moisture. The patient support apparatus 102 can include additional sensors that measure one or more physiological parameters of the patient P such as respiration rate, heart rate, non-invasive blood pressure (NIBP), motion, and weight. Additionally, the patient support apparatus 102 can include sensors that detect patient exit, deterioration, and other conditions relevant to healthcare.

The patient support apparatus 102 is connected to a network 110 that can exchange data between the patient support apparatus 102 and other equipment inside the patient environment 100. Further, the network 110 can connect and exchange data between the patient support apparatus 102 and other systems outside of the patient environment 100 such as a nurse call system 200 and an electronic medical record (EMR) system 300. The network 110 can include any type of wired or wireless connections, or any combinations thereof. The wireless connections can be accomplished using Wi-Fi, ultra-wideband (UWB), Bluetooth, and the like.

The network 110 transfers alerts generated by the patient support apparatus 102 to the nurse call system 200 which communicates with a plurality of devices such as mobile devices 202 (e.g., smartphones, tablet computers, and the like) and workstation monitors 204. As an illustrative example, the nurse call system 200 can transfer the alerts generated by the patient support apparatus 102 to the mobile devices 202 and the workstation monitors 204.

In some examples, the nurse call system 200 is communicatively connected to the mobile devices 202 and the workstation monitors 204 via the network 110. Alternatively, the nurse call system 200 can communicate with the mobile devices 202 and the workstation monitors 204 via wired and/or wireless connections without using the network 110.

The EMR system 300 collects electronic health information of the patient P in a digital format for storage in an electronic medical record (EMR) 302 of the patient P. For example, the EMR system 300 can receive via the network 110 physiological parameter measurements and alerts detected by the patient support apparatus 102 for storage in the EMR 302 of the patient P. As described herein, electronic health records (EHRs) and electronic patient record (EPRs) can be used interchangeably with EMRs.

The EMR system 300 maintains a plurality of EMRs 302 for a plurality of patients who are admitted to the healthcare facility, or who were previously admitted to the healthcare facility. The EMRs 302 can be shared across different health care settings. For example, the EMRs 302 can be shared through network-connected, enterprise-wide information systems or other information networks and exchanges. The EMRs 302 can include a range of data, including demographics, medical history, medication and allergies, immunization status, laboratory test results, radiology images, vital signs, statistics like age and weight, and billing information.

FIG. 2 is an isometric view of the mattress 106 with a cutaway portion exposing an interior of the mattress 106. As shown in FIG. 2, the mattress 106 includes one or more internal structures 122 such as bladders and/or foam layers that provide cushioning and support for the patient P while resting on the patient support apparatus 102.

The mattress 106 further includes an outer cover 124 that surrounds and envelopes the one or more internal structures 122. The outer cover 124 can be made of an impermeable material to protect the one or more internal structures 122 from fluids and moisture.

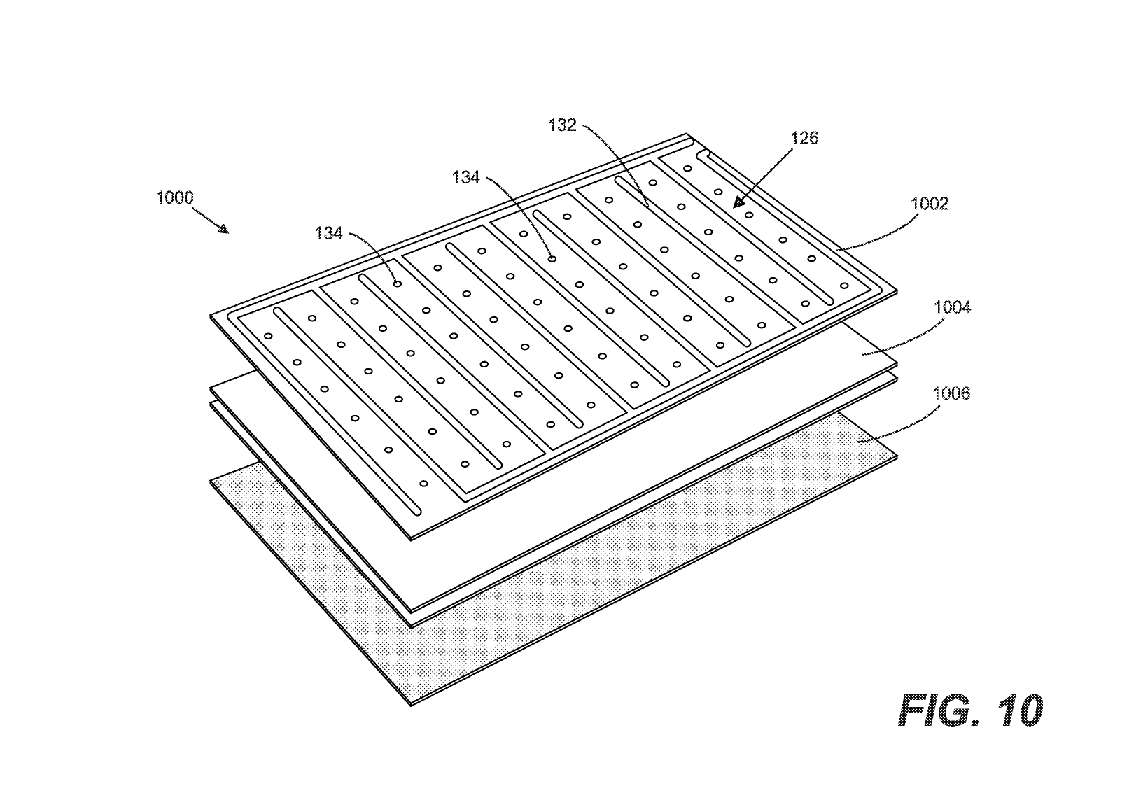

The mattress 106 further includes a sensor 126 fixed between the one or more internal structures 122 and the outer cover 124 such that the sensor 126 is integrated inside the mattress 106. The sensor 126 can be fixed by stitching, glue, tape, or other suitable types of adhesives to secure the sensor 126 between the one or more internal structures 122 and the outer cover 124. In some examples, the sensor 126 is integrated into a microclimate management assembly of the mattress 106, as shown in FIG. 10, which is described in more detail further below.

As described above, the outer cover 124 can be made of an impermeable material such that the sensor 126 does not contact any fluid or moisture that may be present outside of the mattress 106. An electrical connector 128 connects the sensor 126 to the controller 900 housed on the frame 104 of the patient support apparatus 102. As will be described in more detail, data captured by the sensor 126 can be used by the controller 900 of the patient support apparatus 102 to determine whether a presence of fluid outside of the mattress 106 exceeds a threshold.

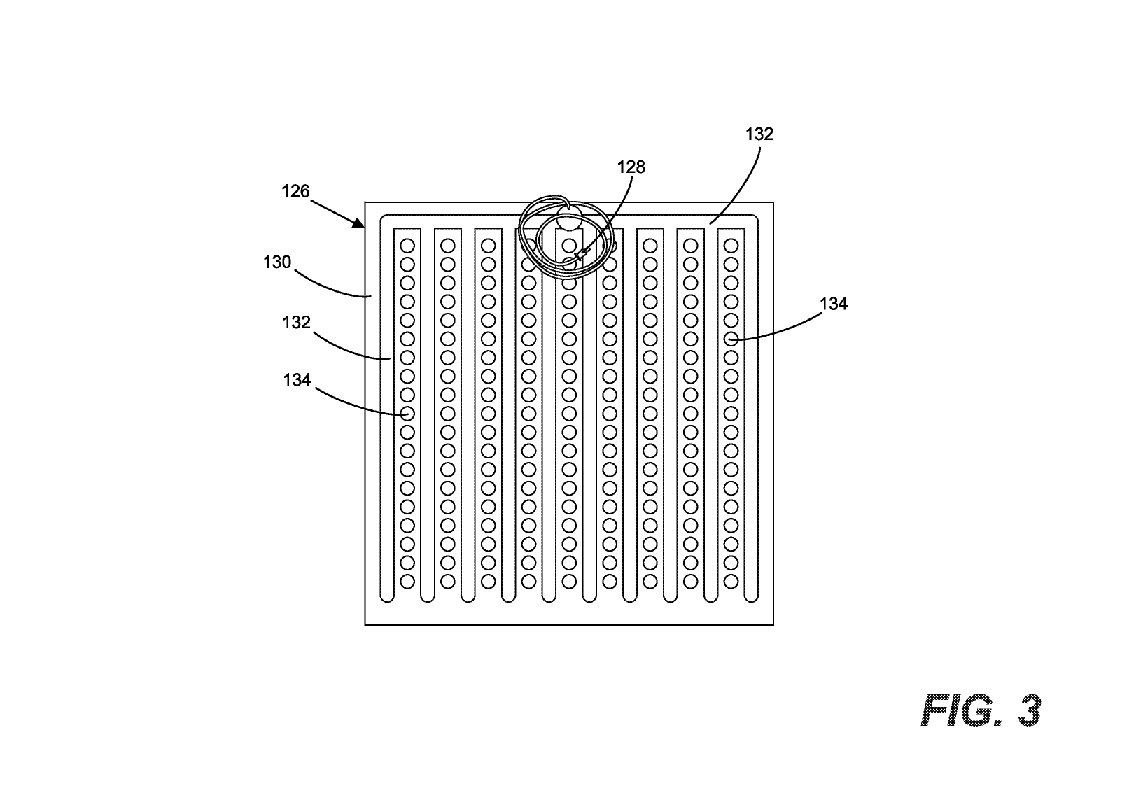

FIG. 3 illustrates an example of the sensor 126 that can be integrated inside the mattress 106. In this example, the sensor 126 includes a substrate 130 and one or more electrically conductive traces 132 arranged on the substrate 130. The one or more electrically conductive traces 132 generate an electrical field that is interrupted by a presence of fluid or moisture outside of the mattress 106 for detection of incontinence.

The interruption of the electrical field is detected without contacting the fluid such that the sensor 126 does not need to contact any fluid or moisture in order to detect a presence of the fluid or moisture. Instead, the sensor 126 is positioned inside the mattress 106 which is surrounded or enveloped by the outer cover 124 which can be made of an impermeable material that prevents the fluid or moisture from entering inside the mattress 106, and thereby prevents the fluid or moisture from contacting the one or more electrically conductive traces 132.

The substrate 130 can be made of a fabric material. For example, the substrate 130 can include a woven textile material. The woven textile material can include a coating to seal the woven textile and the one or more electrically conductive traces 132. As an illustrate example, the substrate 130 can have a length of about 12 inches and a width of about 12 inches.

The one or more electrically conductive traces 132 can include a flexible printed circuit arranged on the substrate 130. For example, the one or more electrically conductive traces 132 can include a flexible ink or other material that is printed on the substrate 130.

The sensor 126 further includes a plurality of holes 134 through the substrate 130. The holes 134 facilitate air flow within the mattress 106 such as between the sensor 126 and the one or more internal structures 122 such as bladders and/or foam layers.

The sensor 126 further includes the electrical connector 128 for connecting the one or more electrically conductive traces 132 to the controller 900 (see FIG. 9) housed on the frame 104 of the patient support apparatus 102. The controller 900 can receive raw data from the sensor 126 and process the raw data to determine whether an interruption of the electrical field generated by the one or more electrically conductive traces 132 indicates a presence of a fluid or moisture outside of the mattress 106 that exceeds a threshold.

When a presence of a fluid or moisture outside of the mattress 106 exceeds the threshold, the controller 900 generates an alert. The alert can be displayed by the patient support apparatus 102 to notify caregivers of patient incontinence. Further, the alert can be communicated from the patient support apparatus 102 to the nurse call system 200 via the network 110. Thereafter, the nurse call system 200 can distribute the alert to appropriate caregivers such as one or more caregivers who are assigned to the patient P. In such examples, the alert can be displayed on one or more of the mobile devices 202 and the workstation monitors 204 to notify the assigned caregivers about the patient P's incontinence.

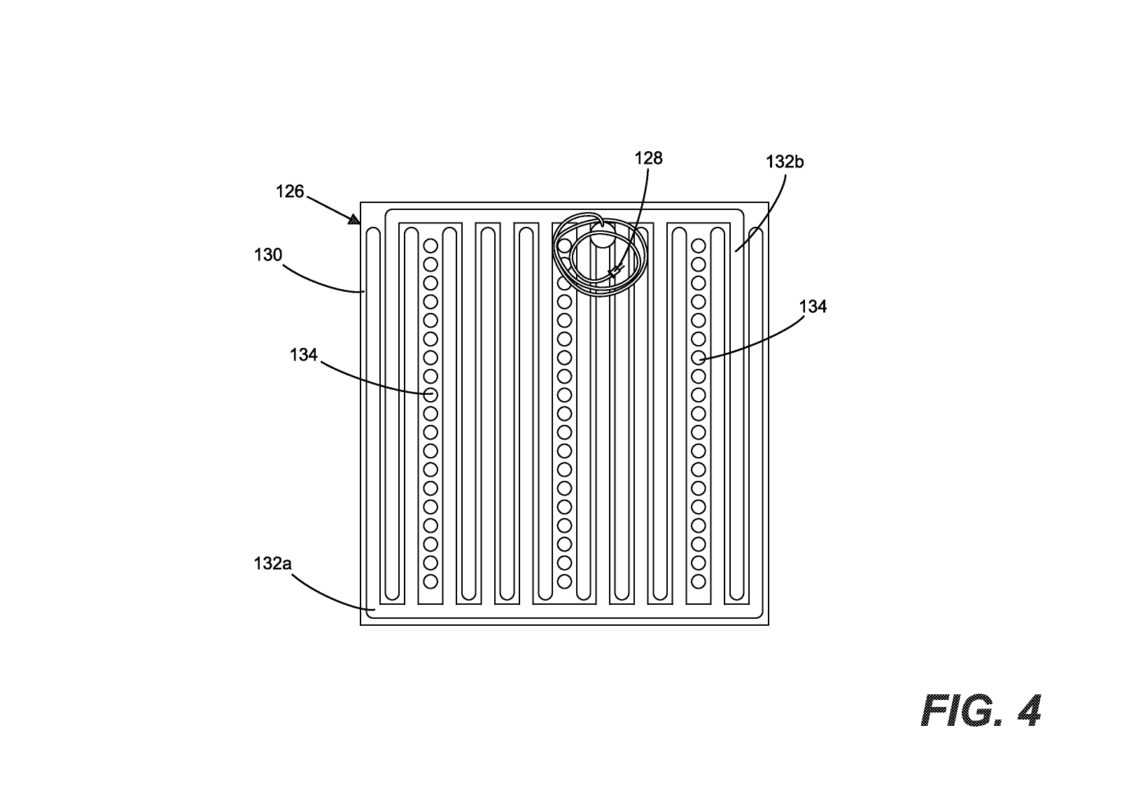

FIG. 4 illustrates another example of the sensor 126 that can be integrated inside the mattress 106. In this example, the sensor 126 similarly includes the electrical connector 128, the substrate 130, the one or more electrically conductive traces 132 arranged on the substrate 130, and the holes 134. The example of the sensor 126 shown in FIG. 4 differs from the example of the sensor 126 shown in FIG. 3 in that the one or more electrically conductive traces 132 and the holes 134 have a different pattern and spacing for generating the electrical field.

For example, in both FIGS. 3 and 4, the one or more electrically conductive traces 132 are arranged in patterns having a plurality of parallel lines on the substrate 130. In FIG. 3, columns of the holes 134 separate the parallel lines of the one or more electrically conductive traces 132. For example, a column of the electrically conductive traces 132 is followed by a column of holes 134, which is followed by a column of the electrically conductive traces 132, which is followed by a column of holes 134, and so on such that the columns of the parallel lines of the electrically conductive traces 132 are separated by the columns of the holes 134.

In FIG. 4, in some instances, the columns of the parallel lines of the one or more electrically conductive traces 132 abut one another such that they are not separated by the holes 134. For example, in FIG. 4, the sensor 126 includes three parallel lines of the electrically conductive traces 132 followed by a column of the holes 134, followed by six parallel lines of the electrically conductive traces 132, followed by a column of the holes 134, followed by six parallel lines of the electrically conductive traces 132, followed by a column of the holes 134, and followed by a three parallel lines of the electrically conductive traces 132.

The example of FIG. 4 includes fewer columns of the holes 134 than the columns of the holes in the example of FIG. 3. For example, the sensor 126 in FIG. 3 includes nine columns of the holes 134 whereas the sensor 126 in FIG. 4 includes three columns of the holes 134.

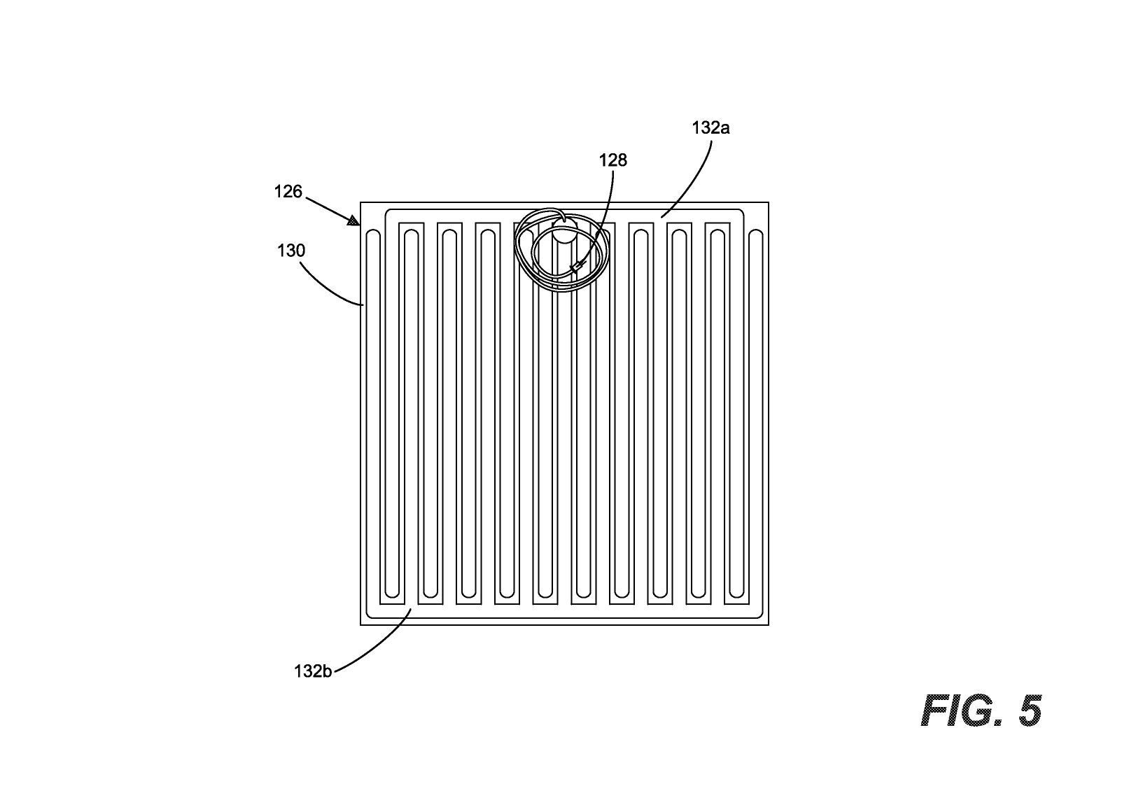

FIG. 5 illustrates another example of the sensor 126 that can be integrated inside the mattress 106. In this example, the sensor 126 similarly includes the electrical connector 128, the substrate 130, and the one or more electrically conductive traces 132 arranged on the substrate 130. The example of the sensor 126 shown in FIG. 5 differs from the examples of the sensor 126 shown in FIGS. 3 and 4 in that the sensor 126 does not include any of the holes 134.

In the example of FIG. 5, the pattern of the electrically conductive traces on the substrate 130 includes a first electrically conductive trace 132a that connects a first set of the parallel lines, and the pattern of the electrically conductive traces on the substrate 130 further includes a second electrically conductive trace 132b that connects a second set of the parallel lines. The second set of parallel lines of the second electrically conductive trace 132b are positioned between the first set of parallel lines of the first electrically conductive trace 132a.

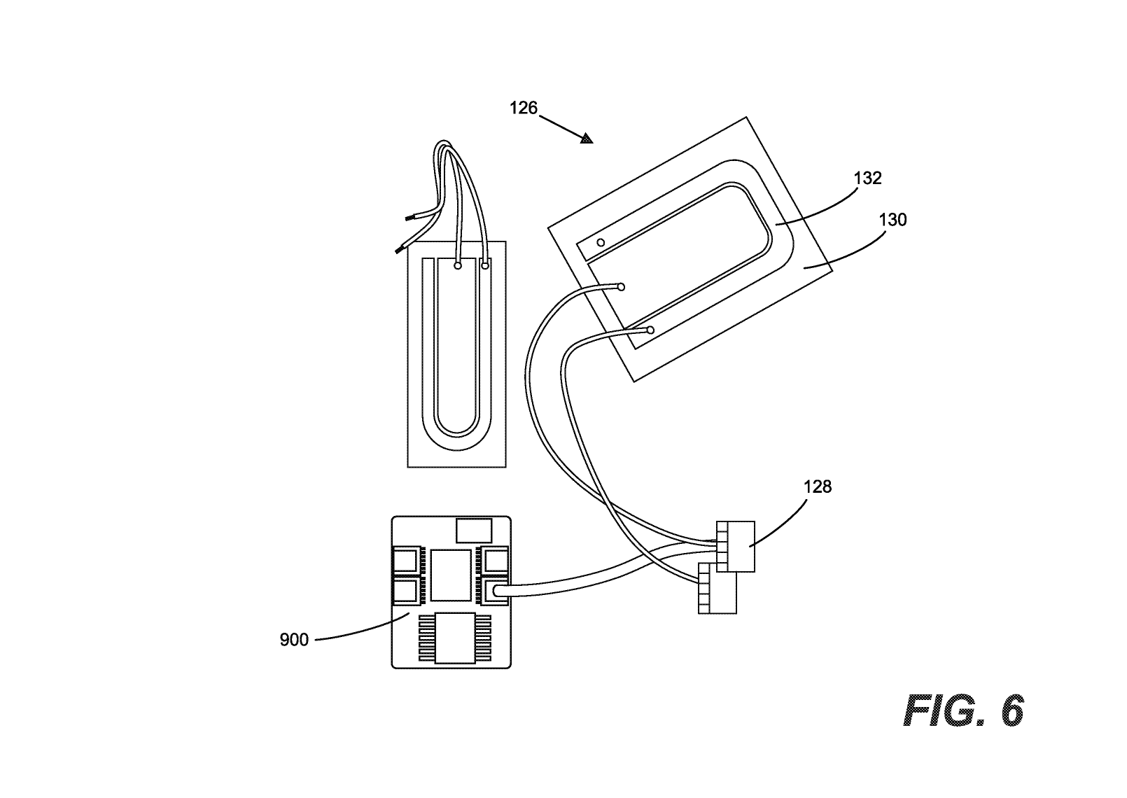

FIG. 6 illustrates another example of the sensor 126 that can be integrated inside the mattress 106. In this example, the sensor 126 similarly includes the electrical connector 128, the substrate 130, and the one or more electrically conductive traces 132 arranged on the substrate 130. The electrical connector is shown connected to the controller 900. In this example, the substrate 130 has a smaller size than in the examples shown in FIGS. 3-5. For example, the substrate 130 can have a size of about 3x4 inches. Also, the one or more electrically conductive traces on the sensor 126 in FIG. 6 have a different pattern than those shown in FIGS. 3-5.

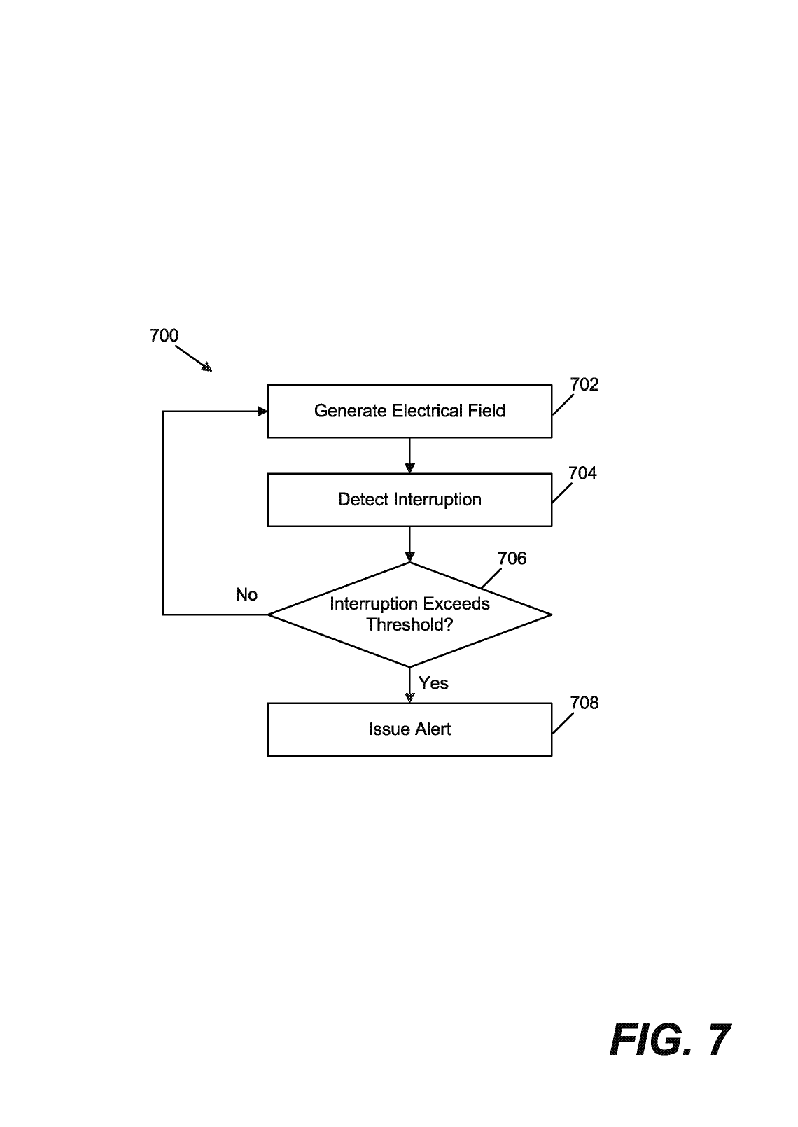

FIG. 7 schematically illustrates an example of a method 700 of detecting incontinence. The method 700 can be performed by the controller 900 of the patient support apparatus 102. The method 700 includes an operation 702 of generating an electrical field using the sensor 126 integrated inside the mattress 106 between the one or more internal structures 122 of the mattress 106 and the outer cover 124 of the mattress 106. As described above, the sensor 126 includes the one or more electrically conductive traces 132 that generate the electrical field.

The method 700 includes an operation 704 of detecting an interruption of the electrical field generated in operation 702. The electrical field can be interrupted by a presence of fluid or moisture outside of the mattress 106. The interruption of the electrical field is detected without the fluid or moisture contacting the sensor 126. The interruption of the electrical field varies based on the quantity of fluid or moisture outside of the mattress 106.

The method 700 includes an operation 706 of determining whether the interruption of the electrical field exceeds a threshold such that the fluid or moisture exceeds a predetermined amount outside of the mattress 106. When operation 706 determines that the interruption of the electrical field exceeds the threshold (i.e., "Yes" in operation 706), the method 700 proceeds to an operation 708 of issuing an alert. Otherwise, when operation 706 determines that the interruption of the electrical field does not exceed the threshold (i.e., "No" in operation 706), the method 700 returns to operation 702 such that operations 702-706 can be repeated until the interruption of the electrical field exceeds the threshold (i.e., "Yes" in operation 706). Operation 706 prevents generating the alert when fluid or moisture is detected but is less than the predetermined amount. This can help to mitigate alarm fatigue.

In operation 708, the alert can be generated on the patient support apparatus 102. For example, a light on the patient support apparatus 102 can be turned on to indicate that fluid or moisture exceeds a predetermined amount outside of the mattress 106. Also, the alert can be communicated from the patient support apparatus 102 to the nurse call system 200 via the network 110. The nurse call system 200 can then send the alert to one or more mobile devices 202 and/or workstation monitors 204 for action by an appropriate caregiver. In some examples, the alert can be stored in the EMR 302 of the patient P via communication from the patient support apparatus 102 over the network 110 to the EMR system 300.

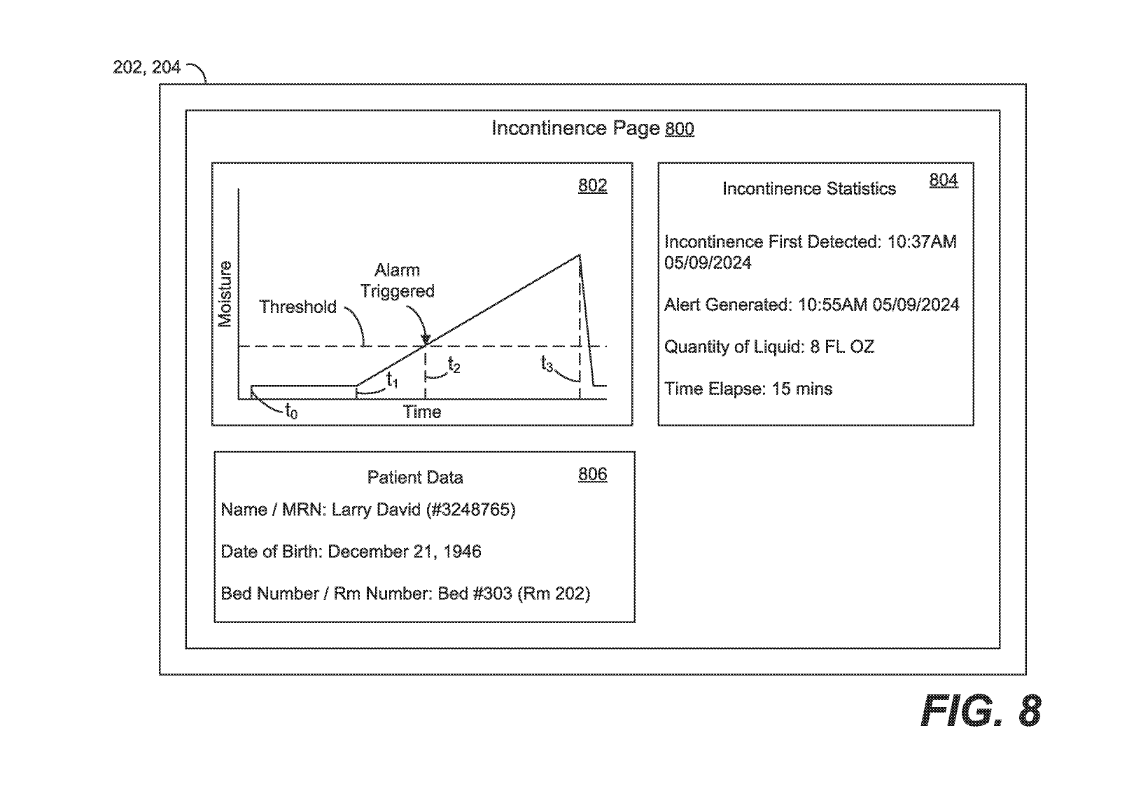

FIG. 8 schematically illustrates an incontinence page 800 that can be generated based on the data collected by the controller 900 of the patient support apparatus 102 from the sensor 126. As described above, the sensor 126 is integrated inside the mattress 106 such that the sensor 126 detects a presence of fluid or moisture without contacting the fluid or moisture.

As shown in FIG. 8, the incontinence page 800 can be displayed on a mobile device 202 or a workstation monitor 204. In some instances, the incontinence page 800 is displayed as an EMR plug-in in the EMR 302 of the patient P stored on the EMR system 300.

In the example illustrated in FIG. 8, the incontinence page 800 includes an incontinence graphical portion 802 that displays fluid or moisture content (Y-axis) over time (X- axis) relative to a threshold for triggering issuance of an alert when the fluid or moisture outside of the mattress 106 exceeds a predetermined amount. In this illustrative example, the fluid or moisture content on the mattress 106 is initially zero at time to such that the mattress 106 is dry. Then, at time ti, the fluid or moisture content is detected due to interruptions of the of the electrical field generated by the sensor 126. Initially, the fluid or moisture content is below the threshold displayed on the incontinence graphical portion 802 such that an alert is not generated.

As further shown in the example of the incontinence graphical portion 802, the fluid or moisture content then gradually increases such that at time t2 the fluid or moisture content exceeds the threshold. Thus, at time t2, the alert is generated for display on the patient support apparatus 102 and/or on one or more of the mobile devices 202 and workstation monitors 204. As described above, this can help to reduce alarm fatigue because the alert is issued only when the fluid or moisture content exceeds a predetermined amount associated with the threshold.

As further shown in the incontinence graphical portion 802, the fluid or moisture content drops at time t3 and returns back to zero. This can occur such as when a caregiver responds to the alert that is generated when the fluid or moisture content exceeds the threshold such as by changing the sheets and linens on the mattress 106 such that the fluid or moisture content is no longer detected by the sensor 126 embedded inside the mattress 106.

The incontinence page 800 can further include an incontinence statistics portion 804 that displays one or more parameters measured based on the interruptions of the electrical field generated by the sensor 126. For example, the incontinence statistics portion 804 can display a time when the fluid or moisture is first detected (time ti), a time when the presence of the fluid or moisture outside of the mattress 106 exceeds the threshold such that the alert is generated (time t2), a time elapsed between issuance of the alert and when the incontinence is addressed by a caregiver (time t₃), and a volume of the fluid or moisture outside of the mattress 106.

As further shown in FIG. 8, the incontinence graphical portion 802 and the incontinence statistics portion 804 can be displayed alongside a patient data portion 806 that can display information such as the patient P's name, medical record number, date of birth, and bed number or room number where the patient P is admitted in the healthcare facility.

FIG. 9 schematically illustrates an example of a controller 900 that can be housed on the frame 104 of the patient support apparatus 102. The controller 900 is communicatively connected to the sensor 126 such that the controller 900 can be used to implement aspects described above. The nurse call system 200, the EMR system 300, the mobile devices 202, and the workstation monitors 204 can include computing devices that have similar components as the components of the controller 900 illustrated in FIG. 9.

As shown in FIG. 9, the controller 900 includes at least one processing device 902, a system memory 908, and a system bus 920 that couples the system memory 908 to the at least one processing device 902. Further, the controller 900 operates in a networked environment using logical connections to devices through the network 110. The controller 900 connects to the network 110 through a network interface unit 904 connected to the system bus 920. The network interface unit 904 may also be utilized to connect to other types of communications networks and devices, including through Bluetooth and Wi-Fi.

The at least one processing device 902 is an example of a processing unit such as a central processing unit (CPU). The at least one processing device 902 can include one or more CPUs. In some examples, the at least one processing device 902 includes one or more digital signal processors, field-programmable gate arrays, and/or other types of electronic circuits.

The system memory 908 includes a random-access memory ("RAM") 910 and a read- only memory ("ROM") 912. Basic input/output logic containing routines to transfer information between elements within the controller 900, such as during startup, is stored in the ROM 912.

The controller 900 can also include a mass storage device 914 that is able to store software instructions and data. The mass storage device 914 is connected to the at least one processing device 902 through a mass storage controller (not shown) connected to the system bus 920. The mass storage device 914 and its associated computer-readable data storage media provide non-volatile, non-transitory storage for the controller 900.

The mass storage device 914 and the RAM 910 can store software instructions and data. The software instructions can include an operating system 916 suitable for controlling the operation of the controller 900. The mass storage device 914 and/or the RAM 910 also store software instructions 918, that when executed by the at least one processing device 902, cause the device to provide the functionality discussed herein.

Although the description of computer-readable data storage media contained herein refers to a mass storage device, it should be appreciated by those skilled in the art that computer- readable data storage media can be any available non-transitory, physical device or article of manufacture from which the device can read data and/or instructions. In certain embodiments, the computer-readable storage media comprises entirely non-transitory media. The mass storage device 914 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

FIG. 10 is an exploded isometric view of an example of a microclimate management assembly 1000 of the mattress 106 that includes an example implementation of the sensor 126 for detecting incontinence. In some examples, the microclimate management assembly 1000 is a structure integrated with the mattress 106. In other examples, the microclimate management assembly 1000 is an accessory of the mattress 106 such as a mattress topper. The microclimate management assembly 1000 can be surrounded by the outer cover 124.

As shown in FIG. 10, the microclimate management assembly 1000 includes several layers such as a first layer 1002 that includes the sensor 126, a second layer 1004 that can include spacer fabric, and a third layer 1006 that can include bottom ticking. The first layer 1002, the second layer 1004, and the third layer 1006 can be fixed together to form the microclimate management assembly 1000 by stitching, glue, heat sealing, or other suitable types of adhesives.

As shown in FIG. 10, the sensor 126 includes the one or more electrically conductive traces 132 and the plurality of holes 134 through the first layer 1002 such that the sensor 126 shown in FIG. 10 is similar to the examples of the sensor 126 shown in FIG. 3 or FIG. 4. The holes 134 facilitate air flow within the microclimate management assembly 1000 such as between the first layer 1002 having the sensor 126 and the second and third layers 1004, 1006.

The various embodiments described above are provided by way of illustration only and should not be construed to be limiting in any way. Various modifications can be made to the embodiments described above without departing from the true spirit and scope of the disclosure.

What is claimed is:

1. A mattress comprising:
one or more internal structures;
an outer cover surrounding the one or more internal structures; and
a sensor fixed between the one or more internal structures and the outer cover such that the sensor is integrated with the mattress, the sensor including:
a substrate;
one or more electrically conductive traces arranged on the substrate, the one or more electrically conductive traces configured to generate an electrical field that is interrupted by a presence of a fluid; and
an electrical connector for connecting the one or more electrically conductive traces to a controller housed on a frame that is configured to support the mattress, wherein an interruption of the electrical field causes the controller to issue an alert when the presence of the fluid outside of the mattress exceeds a threshold.

2. The mattress of claim 1, wherein the outer cover of the mattress is made of an impermeable material such that the interruption of the electrical field is detected without contacting the fluid.

3. The mattress of claim 1, wherein the substrate is made of a fabric material.

4. The mattress of claim 1, wherein the substrate includes a plurality of holes for air flow.

5. The mattress of claim 1, wherein the sensor measures based on the interruptions of the electrical field at least one of a time when the incontinence is first detected, a time when the presence of fluid outside of the mattress exceeds the threshold, a volume of fluid on the mattress, and a time elapsed between issuance of the alert and removal of the fluid from the mattress.

6. The mattress of claim 1, wherein the sensor is integrated with a microclimate management assembly of the mattress.

7. The mattress of claim 6, wherein the sensor is included on a first layer of the microclimate management assembly of the mattress, and the microclimate management assembly of the mattress further includes a second layer of spacer fabric and a third layer of ticking.

8. A method of detecting incontinence, the method comprising:
generating an electrical field using a sensor integrated with a mattress;
detecting an interruption of the electrical field;
determining whether the interruption of the electrical field exceeds a threshold such that a fluid outside of the mattress exceeds a predetermined amount; and
issuing an alert when the interruption of the electrical field exceeds the threshold.

9. The method of claim 8, wherein the interruption of the electrical field is detected without contacting the fluid.

10. The method of claim 8, further comprising:
generating the alert on a frame of a patient support apparatus supporting the mattress.

11. The method of claim 8, further comprising:
communicating the alert to a nurse call system.

12. The method of claim 8, further comprising:
measuring based on the interruption of the electrical field at least one of a time when the incontinence is first detected, a time when the presence of fluid outside of the mattress exceeds the threshold, a volume of fluid outside of the mattress, and a time elapsed between issuance of the alert and removal of the fluid from the mattress.

13. The method of claim 8, wherein the sensor is integrated with the mattress between one or more internal structures of the mattress and an outer cover of the mattress, and the sensor includes one or more electrically conductive traces for generating the electrical field.

14. A sensor for detecting incontinence, the sensor comprising:
a substrate configured to be fixed between one or more internal structures of a mattress and an outer cover of the mattress for integration with the mattress;
one or more electrically conductive traces applied on the substrate, the one or more electrically conductive traces configured to generate an electrical field that is interrupted by a presence of fluid outside of the mattress; and
an electrical connector configured to connect the one or more electrically conductive traces to a controller housed on a frame supporting the mattress, wherein an interruption of the electrical field causes the controller to issue an alert indicating the presence of the fluid outside of the mattress exceeds a threshold volume.

15. The sensor of claim 14, wherein the interruption of the electrical field is detected without contacting the fluid.

16. The sensor of claim 14, wherein the substrate is made of a fabric material.

17. The sensor of claim 14, wherein the substrate includes a plurality of holes for air flow.

18. The sensor of claim 14, wherein the sensor measures based on the interruptions of the electrical field at least one of a time when the incontinence is first detected, a time when the presence of fluid outside of the mattress exceeds the threshold volume, a total volume of fluid detected outside of the mattress, and a time elapsed between issuance of the alert and removal of the fluid from the mattress.

19. The sensor of claim 14, wherein the sensor is a first layer of a microclimate management assembly of the mattress.

20. The sensor of claim 19, wherein the microclimate management assembly further includes a second layer of spacer fabric and a third layer of ticking.
